(19) United States
(12) Patent Application Publication
(10) Pub. No.: US 2005/0209630 A1
(43) Pub. Date: Sep. 22, 2005

(54) Control device for a balloon catheter

(76) Inventors: Walter Lutz, Wertheim (DE); Michael Weiss, Hasloch (DE)

Correspondence Address: DILWORTH & BARRESE, LLP, 333 EARLE OVINGTON BLVD., UNIONDALE, NY, 11553, US

(21) Appl. No.: 11/081,171

(22) Filed: Mar. 16, 2005

(30) Foreign Application Priority Data
Mar. 16, 2004 (DE) 102004 012801.4

(51) Int. Cl. A61M 29/00

(52) U.S. Cl. 606/192; 604/98.01

(57) ABSTRACT

A control device serves to operate a balloon catheter. To improve such a control device, it comprises a housing (6) and a piston-in-cylinder unit (16) releasably connectable to the housing (6).

The invention relates to a control device for a balloon catheter and to a piston-in-cylinder unit for such a control device.

Balloon catheters are used in different medical areas and in other areas. They comprise a pressure line, a housing part and a balloon which can be enlarged or inflated by an internal pressure effect. A liquid is generally used as the pressure medium.

The control device in accordance with the invention serves to operate such a balloon catheter. It serves in particular to supply the balloon catheter with a pressure medium, in particular with a liquid under pressure.

Previously known control devices for balloon catheters are designed as disposable devices. They can only be sterilized under excessively large difficulties and/or costs, or even not at all. A repeated use of the previously known control devices is not possible.

A control and inflation device for a balloon catheter is known from EP 565 045 A1 which comprises a cylinder and a piston longitudinally movable therein.

DE-OS 41 15 683 discloses a liquid displacement and pressure generation apparatus for angioplastic balloon catheters with a quick-release mechanism which permits a fast displacement of a screw piston rod provided with a thread to achieve a pressure increase and subsequently the selective coupling of the mechanism with the threaded surface of the piston rod.

It is the object of the invention to provide an improved control device for a balloon catheter.

This object is solved in accordance with the invention by the features of claim 1. The control device for a balloon catheter comprises a housing and a piston-in-cylinder unit which is releasably connectable to the housing. Prior to use of the control device, the piston-in-cylinder unit can be connected to the housing, in particular inserted into the housing. After use of the control device, the piston-in-cylinder unit can be released from the housing, in particular taken out of the housing. The housing can then be used again. It is preferably designed such that it does not come into contact with the pressure medium, in particular the pressure liquid, serving to operate the balloon catheter. Instead, or additionally, the housing can be designed such that it can be sterilized or such that those parts which come into contact with the pressure medium can be sterilized. An opening for the piston-in-cylinder unit can be provided in the housing. The piston-in-cylinder unit can be inserted into the housing through this opening and be connected to the housing prior to use. After use, the piston-in-cylinder unit can be released from the housing and taken out of the housing through the opening.

Advantageous further developments are described in the dependent claims.

A pressure measuring unit is preferably provided in the housing. In many applications, it is desirable or necessary to measure the pressure in the pressure medium, in particular the pressure liquid, serving the operation of the balloon catheter, with an ongoing or uninterrupted pressure measurement being possibly advantageous or necessary.

In previously known control devices for balloon catheters, the pressure is measured directly in the pressure medium. It is, in contrast, advantageous for the pressure measuring unit to have a pressure transducer which is in active communication with the cylinder and/or with the piston. The active communication can be designed such that the pressure transducer does not come into contact with the pressure medium of the balloon catheter so that the pressure transducer or parts thereof cannot be contaminated by this pressure medium. In this case, the pressure transducer is in indirect active communication with the cylinder and/or with the piston.

It is advantageous for the pressure transducer of the pressure measuring unit to contact the cylinder base. Instead or additionally, it can be advantageous for the pressure transducer to contact the piston, in particular the end of the piston and/or a push rod for the piston. In these cases, it is possible for the pressure transducer to contact the cylinder base or the piston or the end of the piston or the push rod directly. It is, however, also possible for the pressure transducer to contact the cylinder base or the piston or the end of the piston or the push rod via one or more intermediate pieces.

A further advantageous further development is characterized in that the pressure transducer of the pressure measuring unit is made as a membrane. The pressure transducer of the pressure measuring unit can furthermore be made as a toothed rack.

In accordance with a further advantageous further development, the piston-in-cylinder unit can be laid or inserted into the housing. The housing can have an opening through which the piston-in-cylinder unit can be laid or inserted into the housing.

It is advantageous for a push rod to be longitudinally displaceably supported in the housing.

Instead or additionally, a push rod having a thread can be rotatably supported in the housing. It is preferably the longitudinally displaceable push rod. A further advantageous further development is characterized in that a threaded nut is provided in the housing and can be latched into the thread of the push rod and/or can be unlatched from the thread of the push rod. The latching and/or unlatching preferably takes place against a spring force. The use of a threaded nut is in particular advantageous when the push rod is also supported longitudinally displaceably. In this case, the fast feed can take place by a longitudinal displacement of the push rod with an unlatched threaded nut, whereas the fine adjustment can be effected by a rotation of the push rod with a latched threaded nut.

The cylinder base can have a stop plate which can be pushed into a mount of a push rod. The mount can be provided at a part which is connected to the housing, for example at a pressure measuring unit.

The piston preferably has a stop plate which can be pushed into a mount of a push rod. The mount can be provided at a part which is connected to the push rod, for example at a pressure measuring unit.

A further advantageous further development is characterized in that the cylinder has projections with which it can be releasably latched in an opening of the housing. It is advantageous for the latch connection to be made by a bayonet connection.

In accordance with a further advantageous further development of the invention, a pivot lever is pivotably supported at the housing and the piston and/or a push rod can be displaced by it. The pivot lever is preferably arranged in the region of a pistol grip formed at the housing.

It is advantageous for a driver to be provided at the pivot lever. The driver can have a thread which can be brought into engagement with a thread of a push rod.

The piston-in-cylinder unit can be actuated by hand, in particular by the described actuation elements, namely a push rod or a pivot lever. It is, however, also possible to operate the piston-in-cylinder unit by motor power. Accordingly, in accordance with a further advantageous further development, the control device includes a motor to drive the piston-in-cylinder unit.

It is advantageous for the control unit to include a display unit and/or an operating unit. The display unit and/or the operating unit can be integrated with the control device in one single housing. It is, however, also possible to accommodate the display unit and/or the operating unit in separate housings.

The invention furthermore relates to a piston-in-cylinder unit for a control device for a balloon catheter having a cylinder in which a piston is displaceably supported. In accordance with a first solution, the cylinder base has a stop plate which can be pushed into a mount of the housing of the control device or of a part connected thereto, in particular of the housing of a pressure measuring unit. In accordance with a further solution, the piston has a stop plate which can be pushed into a mount of a push rod or of a part connected thereto, in particular of the housing of a pressure measuring unit.

It is advantageous for the cylinder to have projections with which it can be releasably latched in an opening of the housing, with the latch connection preferably taking place by a bayonet connection.

A further advantageous further development is characterized in that the cylinder has a connection stub in the region of the cylinder base.

Embodiments of the invention will be explained in detail in the following with reference to the enclosed drawing. There are shown in the drawing:

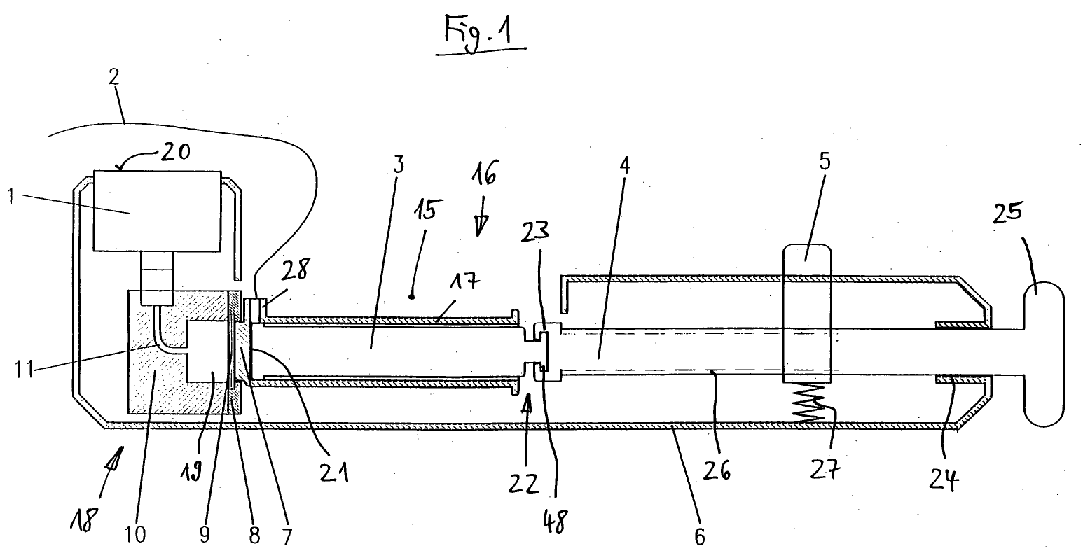

FIG. 1 a control device for a balloon catheter in a schematic side view;

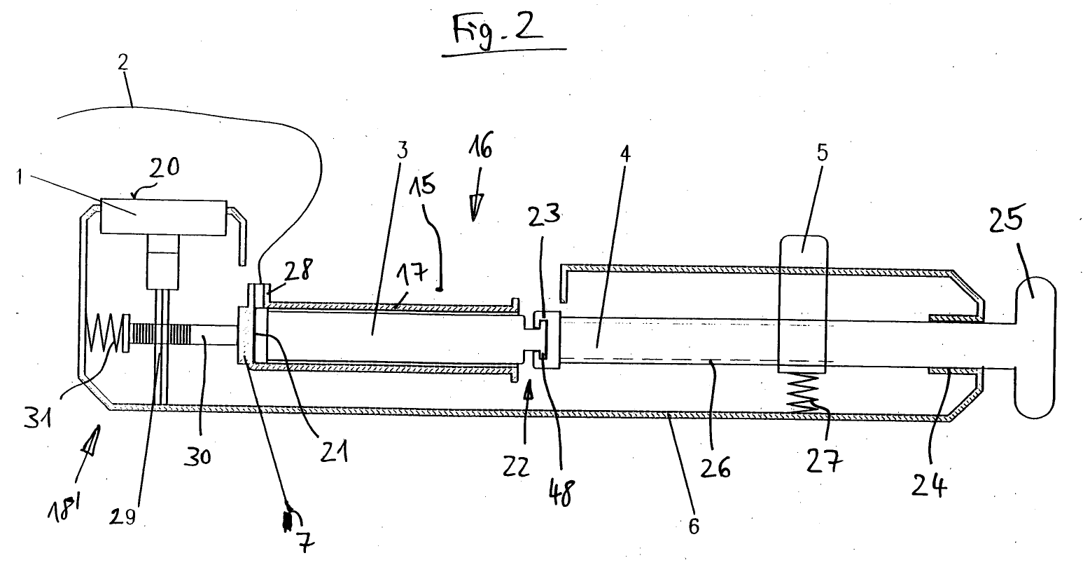

FIG. 2 a modification of the control device in accordance with FIG. 1;

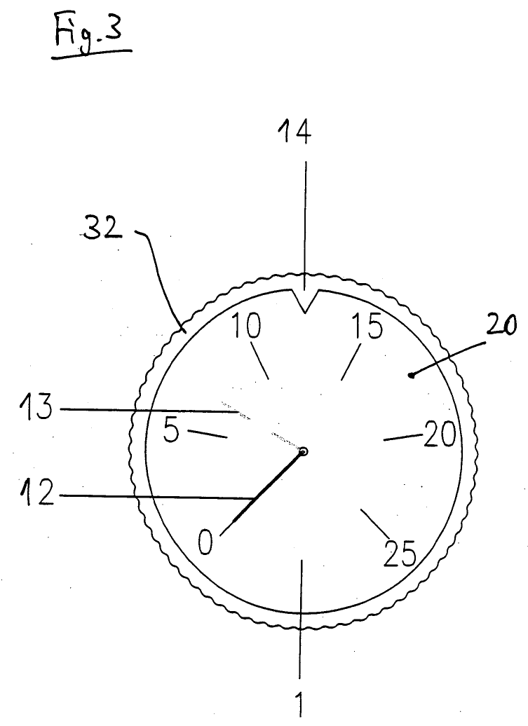

FIG. 3 an adjustment ring for the control device in accordance with FIGS. 1 and 2 in a view from the front;

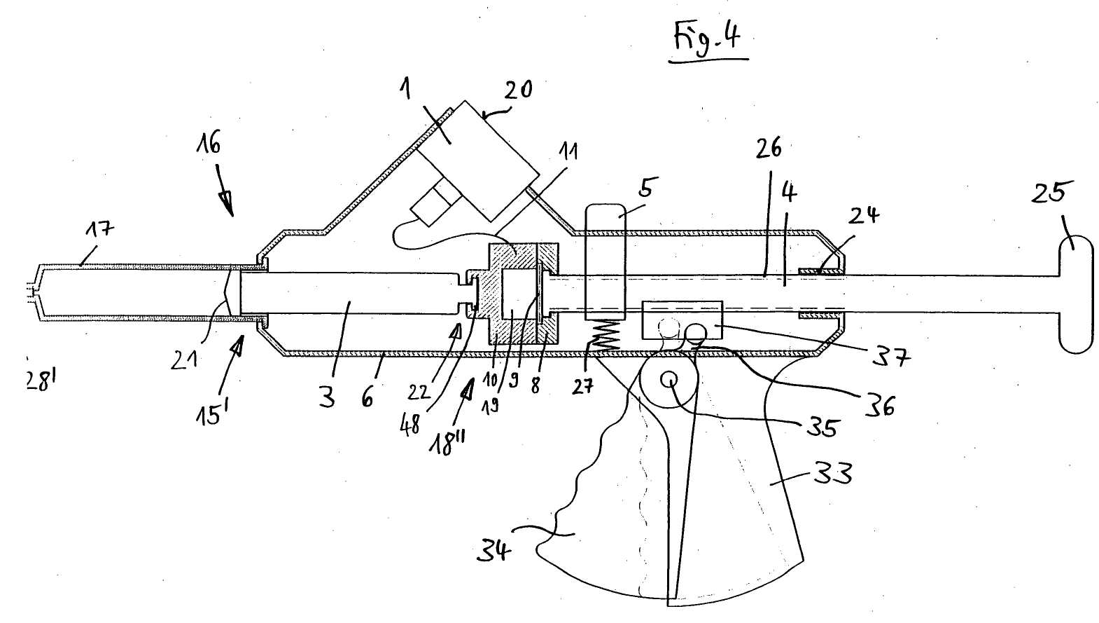

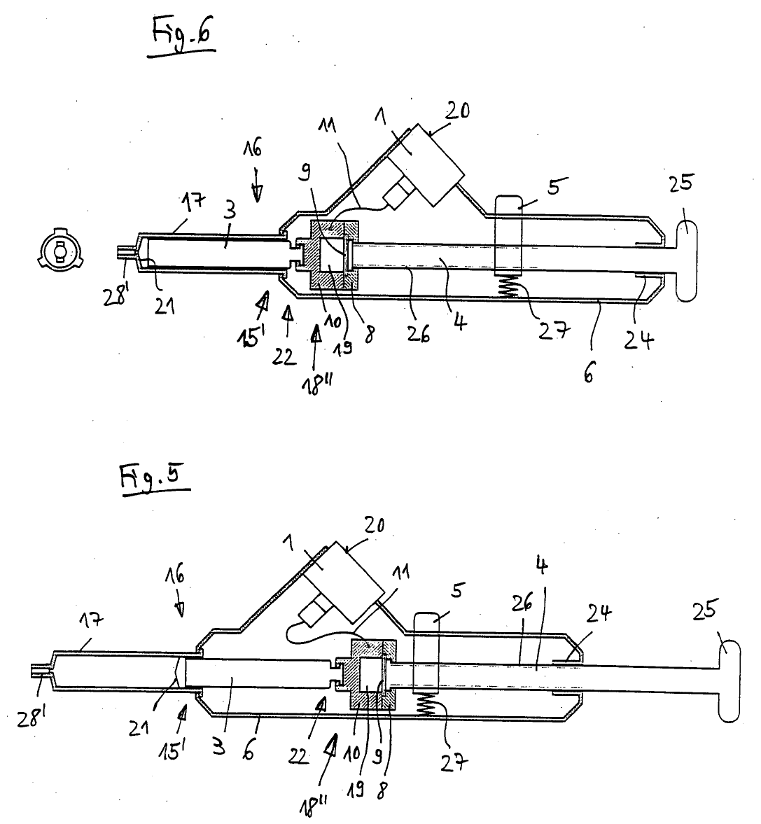

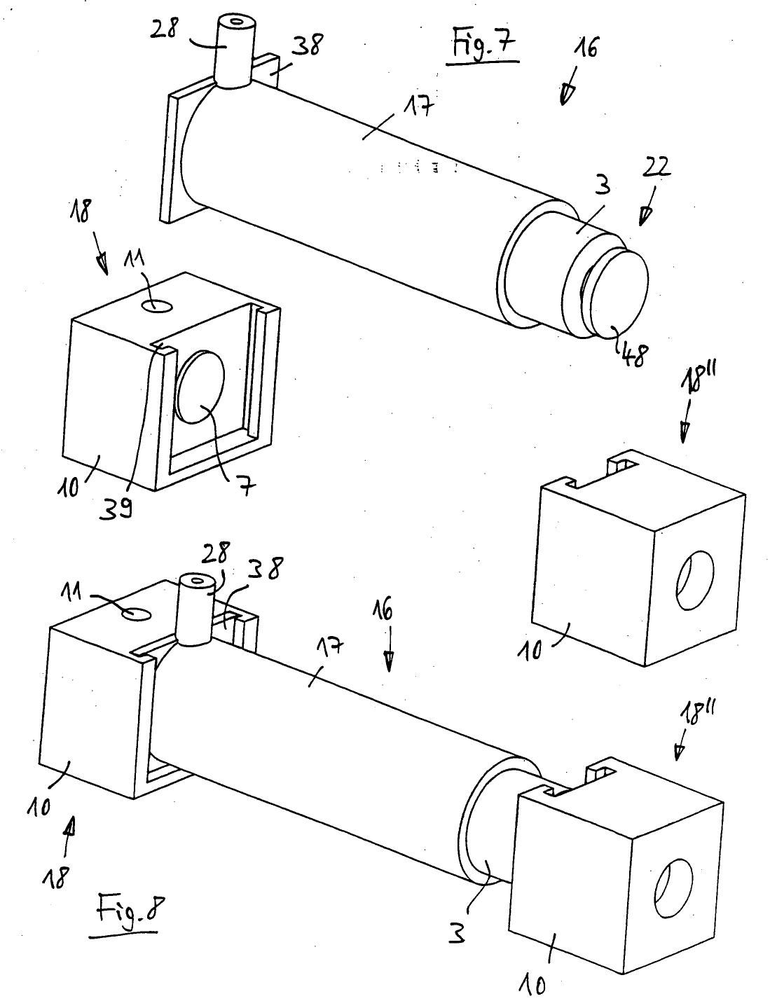

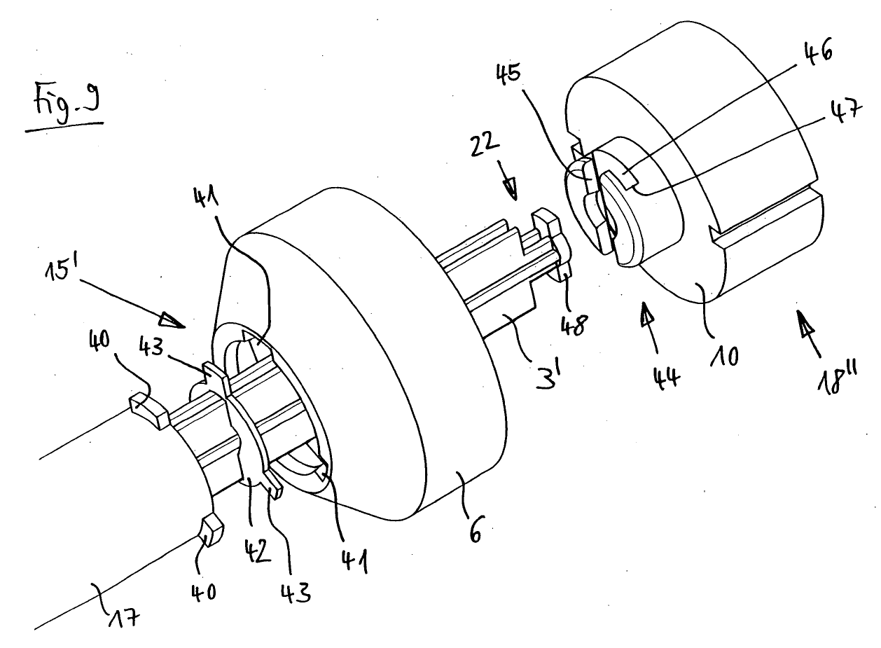

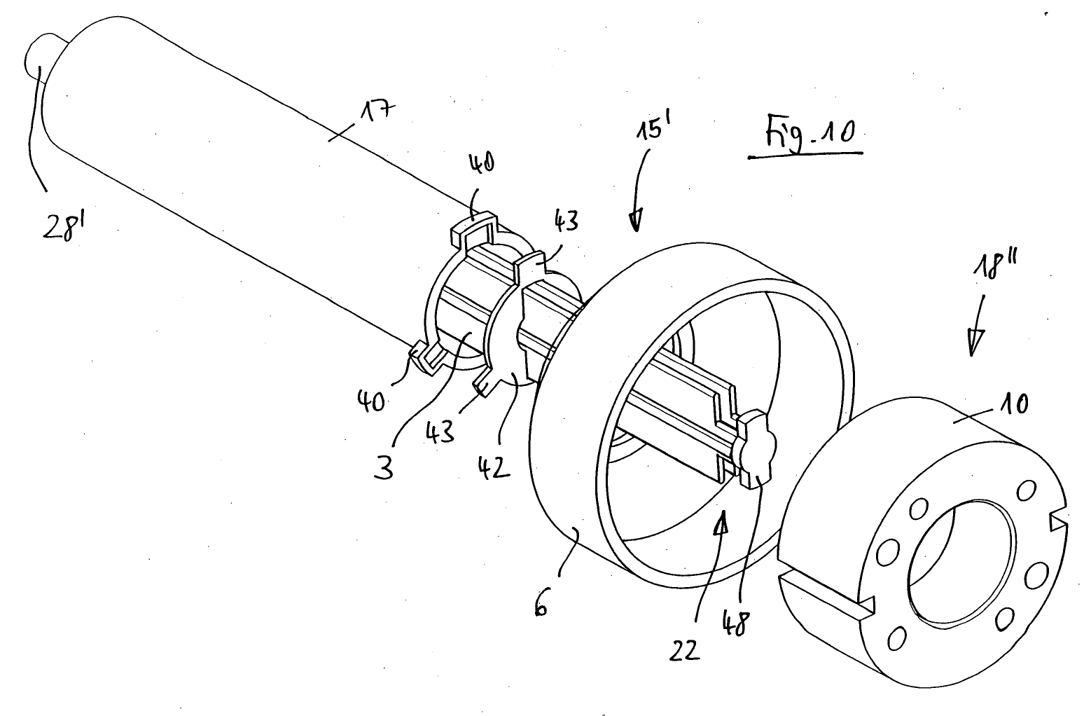

FIG. 4 a modified control device for a balloon catheter in a side view;

FIG. 5 a modification of the control device in accordance with FIG. 4 prior to actuation;

FIG. 6 the control device in accordance with FIG. 5 after actuation;

FIG. 7 a piston-in-cylinder unit and part of a control device prior to the connection in a perspective view;

FIG. 8 the piston-in-cylinder unit in accordance with FIG. 7 after insertion into the control device;

FIG. 9 a modified piston-in-cylinder unit in a perspective view obliquely from the front;

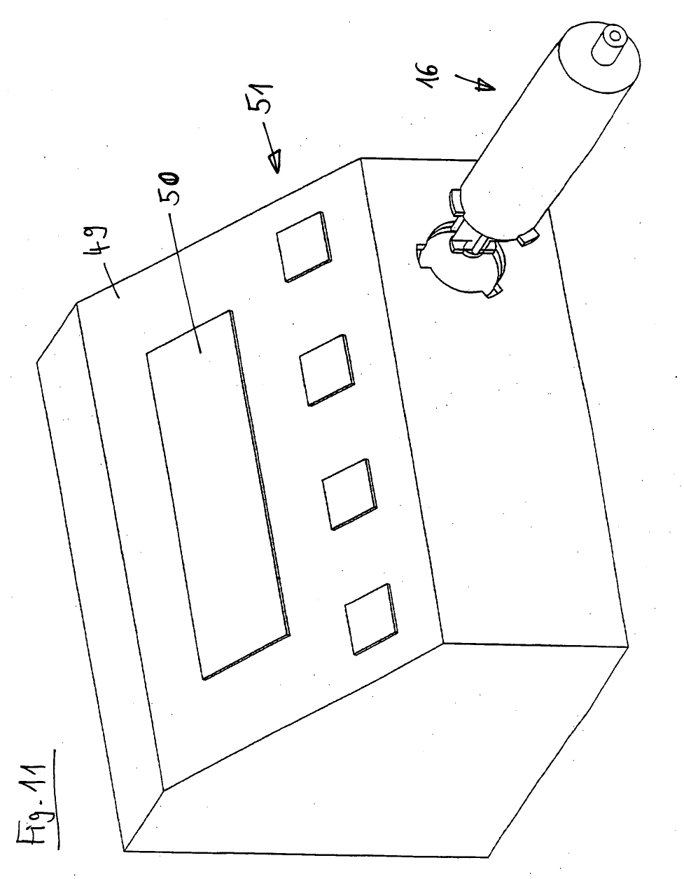

FIG. 10 the piston-in-cylinder unit in accordance with FIG. 9 in a perspective view from behind;

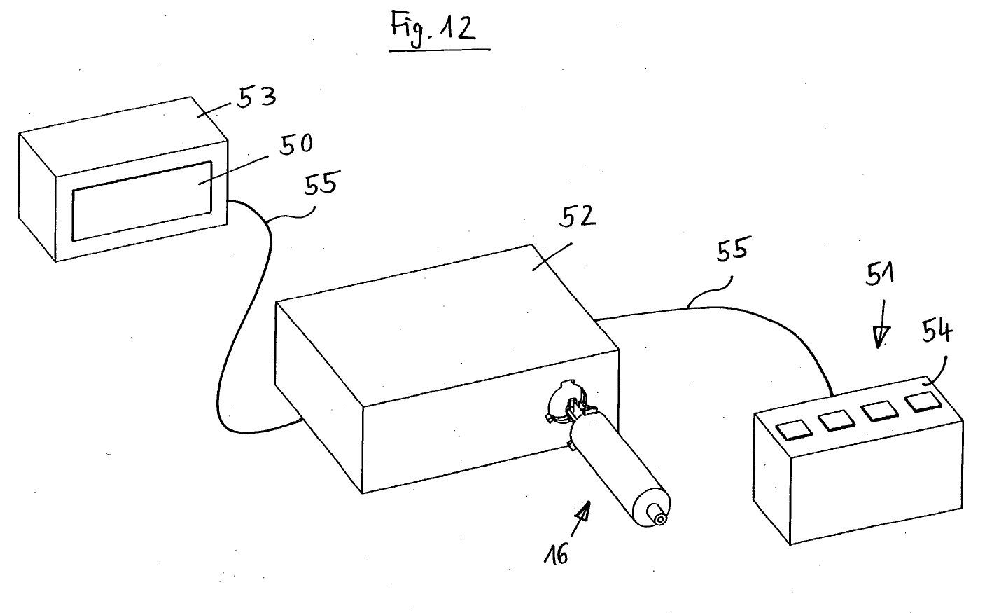

FIG. 11 a control unit with a motor for the drive of the piston-in-cylinder unit; and

FIG. 12 a modification of the control device in accordance with FIG. 11.

The control device shown in FIG. 1 for a balloon catheter comprises a housing 6 and a piston-in-cylinder device 16 which in turn comprises a piston 3 and a cylinder 17. An opening 15 is provided in the housing 6 through which the piston-in-cylinder unit 16 can be inserted into the housing 6 and can be taken out of the housing 6. The piston-in-cylinder unit 16 is releasably connectable to the housing 6.

A pressure measuring unit 18, which is designed as a load cell, is provided-in the housing 6. The pressure measuring unit 18 comprises a housing 10 having a recess 19 whose opening is closed by a membrane 9. The membrane 9 is held by a cover 8 which can be screwed to the housing 10 or can be fastened to the housing 10 by other means. The cylinder base 7, that is the base of the cylinder 17, contacts the membrane 9. It can be thickened in the manner visible from FIG. 1.

A pressure line 11 leads from the recess 19 through the housing 10 to the manometer 1 which is screwed to the housing 10 and whose display surface 20 terminates in a substantially flush manner with an outer surface of the housing 6.

The recess 19 and the pressure line 11 are filled with a hydraulic fluid by which the pressure exerted on the membrane 9 is transmitted to the manometer 1.

The piston 3, which is longitudinally movable in the cylinder 17, has a T-shaped connection piece 22 at its end facing away from the end face 21, said T-shaped connection piece 22 projecting out of the cylinder 17 and its stop plate 48 being releasably connectable to a correspondingly shaped mount 23 of a push rod 4.

The push rod 4 is flush with the piston 3. It is longitudinally displaceably and rotatably supported in a bearing 24 of the housing 6. The push rod 4 is provided with a handgrip 25 at its end facing away from the mount 23 and projecting out of the housing 6. It furthermore has a thread 26.

A threaded nut 5 is provided in the housing 6 and can be latched into the thread 26 of the push rod 4 and unlatched from this thread 26. For this purpose, the threaded nut 5 is biased by a compression spring 27 which is supported on the housing 6, on the one hand, and on the threaded nut 5, at the other. In the state shown in FIG. 1, the threaded nut 5 is latched into the thread 26 of the push rod 4. A feed of the mount 23 of the push rod 4, and thus of the piston 3, can be generated by a rotation at the handgrip 25 of the push rod 4. If a faster feed of the piston 3 should be achieved, the threaded nut 5 can be unlatched from the thread 26. For this purpose, the threaded nut 25 has an end which projects from the housing 6 and which is disposed opposite the compression spring 27. The threaded nut 5 is unlatched from the thread 26 by pressure on this end opposed to the force of the compression spring 27. A feed of the piston 3 in the direction of the piston-in-cylinder unit 16 can now be generated by pressure onto the handgrip 25. The threaded nut 5 can have a device by which it is held in the unlatched position (not shown in the drawing).

In operation, the piston-in-cylinder unit 16 is inserted into the housing 6 through the opening 15. A piston-in-cylinder unit 16 can be inserted into the housing 6 which is not filled with a liquid suitable for the operation of a balloon catheter. In this case, the piston 3 can adopt the position shown in FIG. 1 in which its end face 21 contacts the cylinder base 7. The stop plate 48 of the T-shaped connection piece 22 of the piston 3 is connected to the mount 23 of the push rod 4. A source for balloon catheter liquid is furthermore connected to the support stub 28 of the cylinder 17 (not shown in the drawing). The support stub 28 is located in the region of the cylinder base 7 and its passage opening is partly flush with the cylinder base 7. The piston 3 is pulled up by a pull on the handgrip 25 such that the cylinder 17 is filled with balloon catheter liquid. The connection stub 28 is then connected to the pressure line 2 to the balloon catheter. The operation of the balloon catheter can start. During this operation, the pressure is monitored which is displayed on the indication surface 20 of the manometer 1. The operating pressure of the balloon catheter liquid in the piston-in-cylinder unit 16 acts through the cylinder base 7 on the membrane 9 and through this on the hydraulic liquid in the recess 19 from which this pressure is guided through the pressure line 11 to the manometer 1 and is displayed on the indication surface 20. The measured pressure can also be guided onward. It can in particular be transformed into an electrical signal and supplied to an evaluation and/or display device, in particular to a computer which can also take over the control and/or regulation of the operation of the balloon catheter. This operation can be carried out in accordance with programs which are stored on the computer.

It is, however, also possible to insert a piston-in-cylinder unit 16 into the housing 6 which is already filled with balloon catheter liquid. The opening 15 can be larger for this purpose (not shown in the drawing).

After the end of the operation of the balloon catheter, the pressure line 2 can be released from the connection stub 28. The piston-in-cylinder unit 16 can be taken out of the housing 6 and replaced by a new piston-in-cylinder unit 16.

In FIG. 2, a modification of the system in accordance with FIG. 1 is shown in which coinciding components are provided with the same reference numerals. In contrast to the embodiment in accordance with FIG. 1, in the modification in accordance with FIG. 2 it is not a hydraulic pressure measuring unit which is provided, but rather a mechanical pressure measuring unit 18′ which comprises a toothed rack 30 whose end contacts the cylinder base 7. The other end of the toothed rack 30 is supported at the housing 6 via a compression spring 31. The teeth of the toothed rack 30 engage into corresponding teeth of a toothed shaft 29 which extends at right angles to the toothed rack 30 and is connected to a transmission of a manometer 1 which displays the measured pressure at its face 20. In the modification in accordance with FIG. 2, the pressure measuring unit 18′ comprises a pressure transducer which contacts the cylinder base 7, namely the toothed rack 30.

FIG. 3 shows the face 20 of the manometer 1 which is surrounded by a rotatable adjustment ring 32. The adjustment ring 32 comprises an inwardly facing marking 14 which can be set to a pre-determined pressure, for example to a desired pressure or to a maximum permitted pressure. In the example selected to a pressure of 12.5 bar.

The indicator 12 initially indicates a pressure of 0 bar. In the position 13, a pressure of 6.5 bar is indicated. The marking 14 can be provided with a warning device to trigger an acoustic and/or visual and/or other alarm on the reaching of the pressure set by this marking.

In FIG. 4, a further modification of a control device for a balloon catheter is shown in which components which coincide with those of the embodiments in accordance with FIGS. 1 and 2 are provided with the same reference numerals. In the embodiment in accordance with FIG. 4, the pressure measuring unit 18″ is connected to the piston 3.

It is disposed between the piston 3 and the push rod 4. The pressure measuring unit 18″ otherwise coincides with that in accordance with FIG. 1. It comprises a membrane 9 which is clamped to the housing 10 by a cover 8. Hydraulic fluid is located in the recess 19 and is led through the pressure line 11 to the manometer 1 which displays the measured pressure on the indication surface 20 which essentially terminates flush with an outer surface of the housing 6.

In the embodiment in accordance with FIG. 4, the end face of the push rod 4 facing the piston 3 contacts the membrane 9 of the pressure measuring unit 18″. The operating pressure of the balloon catheter liquid located in the cylinder 17 is transmitted in this manner to the membrane 9 and to the hydraulic fluid located in the recess 19.

The end of the push rod 4 facing the piston 3 is made in a T shape. It is disposed in a corresponding recess inside the cover 8 by which it is held there. The connection between the piston 3 and the push rod 4 is established by the pressure measuring unit 18″.

The cylinder 17 is provided at its front end with a central connection stub 28′ to which the pressure line leading to the balloon catheter can be connected (not shown in the drawing).

The housing 6 is provided in the rear lower region with a pistol grip 33 at which a pivot lever 34 is pivotably supported. The pivot axis 35 is located in the upper front region of the pistol grip 33, somewhat below the lower side of the housing 6. The pivot lever 34 has an actuation lever 36, whose cylindrical or spherical end region engages into a corresponding recess in a driver 37, on the side disposed opposite the pivot axis 35. An internal thread is worked into the upper side of the driver 37 and corresponds to the external thread of the push rod 4, but extends over a peripheral region of a maximum of 180°. The pivot lever 34 is biased by a spring (not shown in the drawing) such that it adopts the position drawn in a solid line in FIG. 4.

When the pivot lever 34 is pressed against this spring bias, it is rotated around the pivot axis 35 in a counterclockwise direction. The end of the actuation lever 36 moves slightly upwardly and in the direction toward the piston 3. The partial internal thread of the driver 37 hereby comes into engagement with the external thread of the push rod 4 and the push rod 4 pushes the piston 3 into the cylinder 17. The pressure arising thereby is taken up at the membrane 9 and displayed by the manometer 1. To allow the movement of the push rod 4, the threaded nut 5 must be pressed against the force of the spring 27 to unlatch the internal thread of the threaded nut 5 from the external thread 26 of the push rod 4. Otherwise, the push rod 4 is blocked; it cannot be moved in the longitudinal direction by pressing the pivot lever 34.

The engagement of the driver 37 into the push rod 4 can be made such that a repetitive actuation is possible. A feed of the piston 3 is then generated by every actuation of the pivot lever 34 counter clockwise. If the pivot lever 34 is released so that it moves in a clockwise direction, no return movement of the push rod 4 takes place in this case.

In the embodiment in accordance with FIG. 4, the piston-in-cylinder unit 16 is inserted into the housing 6 through an opening 15′ which essentially corresponds to the outer peripheral surface or the external diameter of the cylinder 17.

FIG. 5 shows the control device in accordance with FIG. 4, however without a pistol grip 33, in the position prior to the start of operation of the balloon catheter in which the piston 3 is located in the retracted position. During the operation of the balloon catheter, the piston 3 is moved into the cylinder 17, that is in the direction to the left in the representation of FIG. 5, until the position shown in FIG. 6 is adopted. During this operation, the pressure in the balloon catheter fluid, that is inside the cylinder 17, is measured continuously, with the pressure transducer being formed by the membrane 9.

FIG. 7 shows a piston-in-cylinder unit 16 which essentially corresponds to that of FIGS. 1 and 2 and in which coinciding parts are provided with the same reference numerals. The cylinder base 7 is provided with a square stop plate 38 which can be pushed into a correspondingly designed mount 39 of the housing 10 of the pressure measuring unit 18. The mount 39 comprises two parallel grooves in which the edges of the square stop plate 38 are received.

FIG. 8 shows the piston-in-cylinder unit 16 in the pushed-in state in which the rear of the square stop plate 38 contacts the front side of the membrane 7.

In FIG. 7, a pressure measuring unit 18″ is furthermore shown which essentially corresponds to that of FIGS. 4 to 6. For reasons of a simplified drawing representation, both the pressure measuring unit 18 and the pressure measuring unit 18″ are shown in FIGS. 7 and 8. In a realized device, it is advantageous to use only one of these two pressure measuring units 18 or 18″. The housing 10 of the pressure measuring unit 18″ comprises a mount which essentially corresponds to that of the pressure measuring unit 18 and into which the stop plate 48 of the T-shaped connection piece 22 of the piston 3 can be pushed. The mounted position is shown in FIG. 8.

FIGS. 9 and 10 show part of the embodiment in accordance with FIGS. 4 to 6, with coinciding parts being provided with the same reference numerals. The cylinder 17 is provided with projections 40 at its rear end. Corresponding cut-outs 41 are provided at the rim of the opening 15′ of the housing 6. The arrangement and/or design of the projections 40 and cut-outs 41 is made such that the cylinder 17 can only be pushed into the opening 15′ in the housing 6 in a specific position. Peripheral grooves which extend over a specific angular range and whose ends form stops are located behind the cut-outs 41 so that the cylinder 17 can be pushed into the opening 15′ and can be fixed in the manner of a bayonet connection by a subsequent rotation.

The piston 3′ has an elongated cross-section. A rotational security 42 is longitudinally displaceably supported on the piston 3′ and its inner cut-out corresponds to the outer contour of the piston 3′ and its outer contour corresponds to that of the cylinder 17 in the region of the projections 40. After the pushing of the cylinder 17 into the opening 15′, the projections 43 of the rotational security 42 corresponding to the projections 40 come to rest in corresponding cut-outs inside the housing 6 so that the piston 3′ is secured against rotation with respect to the housing 6 in this position.

The T-shaped connection piece 22 of the piston 3′ can be pushed into a mount 44 in the housing 10 of the pressure measuring unit 18″ and can be held there. For this purpose, the mount 44 comprises a longitudinal slot 45 through which the stop plate 48 of the T-shaped connection piece 22 ca be pushed. After passing through the longitudinal slot 45, it can be held in place in the manner of a bayonet connection by a rotation in the peripheral groove 46 located behind it up to the stop 47.

The possibility exists by the invention of realizing a re-sterilizable control device for a replaceable cylinder-in-piston unit for the inflation and/or deflation of balloon catheters. The piston-in-cylinder unit can be removed after use and the control device, which has not come into contact with the balloon catheter fluid, can be sterilized. Only the cylinder, the piston and the pressure line to the balloon catheter come into contact with the balloon catheter fluid. The membrane 9 is made of resilient material, in particular of plastic, for example PTFE, or of metal.

The measurement of the pressure in the balloon catheter fluid does not take place by a measurement in the cylinder, but by an indirect measurement by a membrane (hydraulically) or mechanically or electronically. The pressure measurement can take place by a pressure pick-up at the cylinder base or at the piston or at a push rod for the piston. It is possible to realize the pressure pick-up in or at the thread of the push rod. No internal pressure measurement is carried out (at the interior of the cylinder directly at the balloon catheter fluid), but an external pressure measurement is carried out.

FIG. 11 shows a control device having a housing 49 in which a motor, namely an electric motor, is provided to drive the piston-in-cylinder unit 16. The control device furthermore includes a display unit 50 and an operating unit 51 which includes a plurality of operating buttons. A total of four operating buttons are provided, and indeed for a slow and a fast backward movement of the piston. However, a different button assignment is also possible. Furthermore, more than or fewer than four operating buttons can also be provided. As can be seen from FIG. 11, the cylinder of the piston-in-cylinder unit is latched to the housing 46. The electric motor is coupled to the piston. It then actuates the piston. The display unit 50 and the operating unit 51 are integrated into the housing 49 of the control device.

In the modification shown in FIG. 12, the control device includes a first housing 52 in which a motor is provided to drive the piston-in-cylinder unit 16, a second housing 53 in which the display unit 50 is provided and a third housing 54 in which the operating unit 51 is provided. The display unit 50 and the operating unit 51 or their housings 51, 54 are connected to the housing 52 of the control device by cables 55.

1. A control device for a balloon catheter,
characterized by
a housing (6) and a piston-in-cylinder unit (16, 16′) releasably connectable to the housing (6).

2. A control device in accordance with claim 1, wherein the pressure measuring unit (18, 18′, 18″) is provided in the housing (6).

3. A control device in accordance with claim 2, wherein the pressure measuring unit (18, 18′, 18″) has a pressure transducer (9, 30) which is in active communication with the cylinder (17) and/or the piston (3).

4. A control device in accordance with claim 2, wherein the pressure transducer of the pressure measuring unit contacts the cylinder base (7) and/or the piston (3) and/or a push rod (4).

5. A control device in accordance with claim 2, wherein the pressure transducer of the pressure measuring unit is made as a membrane (9) and/or as a toothed rack (30).

6. A control device in accordance with claim 1, wherein the piston-in-cylinder unit (16, 16′) can be laid or inserted into the housing (6).

7. A control device in accordance with claim 1, wherein a push rod (4) is longitudinally displaceably supported (24) in the housing (6).

8. A control device in accordance with claim 1, wherein a push rod (4) having a thread (26) is rotatably supported (24) in the housing (6), wherein a threaded nut (5), which can be latched into the thread (26) of the push rod (4) and/or can be unlatched from the thread (26) of the push rod (4) and/or can be unlatched from the thread (26) of the push rod (4), is preferably provided in the housing (6).

9. A control device in accordance with claim 1, wherein the cylinder base (7) has a stop plate (38) which can be pushed into a mount (39) of the housing (6) of the control device or of the housing (10) of the pressure measuring unit (18, 18′).

10. A control device in accordance with claim 1, wherein the piston (3) has a stop plate (48) which can be pushed into a mount (23) of a push rod (4) or of the housing (10) of the pressure measuring unit (18″).

11. A control device in accordance with claim 1, wherein the cylinder (17) has projections (40) with which it is releaseably latchable in an opening (15′) of the housing (6).

12. A control device in accordance with claim 1, wherein a pivot lever (34) by which the piston (3) and/or a push rod (4) can be displaced is pivotably supported at the housing (6), wherein a driver (37) is preferably provided at the pivot lever (34).

13. A control device in accordance with claim 1, characterized by a motor to drive the piston-in-cylinder unit (16).

14. A control device in accordance with claim 1, characterized by a display unit (5) and/or by an operating unit (51).

15. A piston-in-cylinder (17) in which a piston (3) is displaceably supported,
characterized in that
the cylinder base (7) has a stop plate (38) which can be pushed into a mount (39) of the housing (6) of the control device or of a part connected thereto, in particular of the housing (10) of a pressure measuring unit (18, 18′″).

16. A piston-in-cylinder unit for a control device for a balloon catheter having a cylinder (17) in which a piston (3) is displaceably supported,
characterized in that
the piston (3) has a stop plate (48) which can be pushed into a mount (23) of a push rod (4) or of a part connected thereto, in particular of the housing (10) of a pressure measuring unit (18″).

17. A piston-in-cylinder unit in accordance with claim 15, wherein the cylinder (17) has projections (40) with which it can be releasably latched in an opening (15′) of the housing (6).

18. A piston-in-cylinder unit in accordance with claim 15, wherein the cylinder (17) has a connection stub (28) in the region of the cylinder base (7).

19. A piston-in-cylinder unit in accordance with claim 16, wherein the cylinder (17) has projections (40) with which it can be releaseably latched in an opening (15′) of the housing (6).

20. A piston-in-cylinder unit in accordance with claim 17, wherein the cylinder (17) has a connection stub (28) in the region of the cylinder base (7).
